(19) United States
(12) Patent Application Publication
(10) Pub. No.: US 2023/0363703 A1
(43) Pub. Date: Nov. 16, 2023

(54) SYSTEM AND METHOD INCLUDING AFFECT IN PAIN LEVEL RECOGNITION

(71) Applicant: University of South Florida, Tampa, FL (US)

(72) Inventors: Shaun Joseph Canavan, Tampa, FL (US); Md Taufeeq Uddin, Tampa, FL (US); Ghadh Alzamzmi, Rockville, MD (US)

(21) Appl. No.: 18/360,407

(22) Filed: Jul. 27, 2023

Related U.S. Application Data
(63) Continuation of application No. PCT/US22/13871, filed on Jan. 26, 2022.
(60) Provisional application No. 63/142,010, filed on Jan. 27, 2021.

(51) Int. Cl. A61B 5/00; G06V 40/16; G16H 50/70; A61B 5/0205

(52) U.S. Cl. CPC A61B 5/4824; G06V 40/174; A61B 5/0077; A61B 5/7267; G16H 50/70; A61B 5/02055

(57) ABSTRACT

A system and method for pain level recognition using an automated approach which incorporates a pain-affect dataset comprising bioVid pain and bioVid emotion datasets for the assessment of patient pain in clinical settings where patients often experience other affect states, such as anger and anxiety, in addition to pain.

### CROSS-REFERENCE TO RELATED APPLICATIONS

This application is a continuation of and claims priority to PCT International Patent Application No. PCT/US2022/013871, filed Jan. 26, 2022, entitled “SYSTEM AND METHOD INCLUDING AFFECT IN PAIN LEVEL RECOGNITION”, which claims benefit of U.S. Provisional Patent Application No. 63/142,010, filed Jan. 27, 2021, and entitled “SYSTEM AND METHOD FOR ACCOUNTING FOR AFFECT IN PAIN ASSESSMENT”, which is herein incorporated by reference in its entirety.

### BACKGROUND OF THE INVENTION

Automated pain assessment is an essential component in clinical settings to ensure prompt intervention and appropriate treatment. Depending on the context, relying on verbal methods, such as pain scales and questionnaires, for pain assessment may not be objective, reliable, actionable, and scalable. For instance, babies and unconscious patients are unable to provide verbal responses, while verbal responses from a mentally impaired adult may be unreliable.

Publicly available pain datasets, such as UNBC-Mcmaster Pain dataset, bioVid pain dataset and MIntPain dataset, focus on pain and neutral states. Consequently, existing studies do not consider the presence and impact of other affect states while assessing pain. Prior research has discussed that the recognition of the level of pain after the disentanglement of pain from emotions using physiological and behavioral data, and their combination, is an unanswered question.

Current automated pain assessment approaches can be divided broadly into pain detection and pain estimation. Detection approaches detect the presence of pain, while estimation approaches estimate the level of pain compared to a neutral state. Some studies also incorporate pain as a category in a categorical emotion recognition framework. To assess self-reported, observer-reported and expert-reported pain, existing studies utilized different modalities including physiological, brain activity, visual, and fusion of them. These data are analyzed to extract pain-relevant features that are used with different machine learning models such as spatial model, temporal model, person-specific model, and person independent model. Efforts are currently underway to develop machine learning models for automatic pain assessment and management in clinical settings. While these models provide standardized assessment, support continuous monitoring, and allow prompt pain intervention, one limitation is that they are designed and trained only on pain and neutral states.

While there has been significant progress in automated pain assessment, existing pain assessment approaches are limited in comparing pain to only a neutral state (baseline). This approach can lead to the failure of these approaches to obtain the desired outcome in real-world settings where patients are likely to be in multiple affect states aside from being in pain or neutral. For example, it has been reported that hospitalized patients undergo a wide range of affects including anger, depression, anxiety in addition to the pain affect. In this context, affect or affect state refers to the underlying experience of feeling, emotion or mood that a patient is experiencing. These affect states can occur as a result of disease stress, financial concerns, insufficient privacy, and unsatisfactory treatment. Patients can also experience different positive affect states, such as comfort and joy, as a result of interaction with family and caregivers.

It also has been reported that hospitalized neonates experience anger, hunger, and discomfort in addition to pain. Non-pharmacological techniques, such as kangaroo care, musical ternary and breastfeeding, are used to mitigate these affects and provide comfort. Another relevant point is that pain has both physiological and psychological responses, such as the association of pain with emotions and psychiatric disorders.

Accordingly, what is needed in the art is an improved system and method for automated pain assessment that is applicable in a real-world setting.

### SUMMARY OF THE INVENTION

In various embodiments, the present invention provides a system and method that incorporates affect when assessing pain to provide a robust assessment in real-world settings. A new pain-affect dataset is provided and referred to as to as bioVid pain-affect, which allows for the development of a reliable and robust pain assessment model that considers multiple affect states commonly experienced by patients (e.g., sadness and fear) in clinical settings.

In one embodiment, the present invention provides a computer-implemented method for identifying a pain level of a patient of interest, which includes, establishing a pain-affect dataset by merging a pain dataset comprising data acquired from a plurality of patients in response to a stimulus for eliciting pain with an affect dataset comprising data acquired from the plurality of patients in response to a stimulus to elicit a non-pain affect state in the plurality of patients. The method further includes, training a neural network model using the established pain-affect dataset, monitoring a patient of interest with an image capture device to capture image data of a face of the patient of interest, collecting one or more biopotential signals of the patient of interest and applying the trained neural network model to the captured image data of the patient of interest and to the one or more biopotential signals collected from the patient of interest to identify a pain level of the patient of interest.

In particular, the non-pain affect state may include amusement, anger, disgust, fear and sadness.

In one embodiment, the pain dataset may be a bioVid pain dataset including image data and data collected from electrodermal activity (EDA), electrocardiogram (ECG), electromyogram (EMG) of a trapezius muscle, EMG of a corrugator muscle and EMG of a zygomaticus muscle of the plurality of patients, the non-pain affect dataset may be a bioVid affect dataset including image data and data collected from electrodermal activity (EDA), electrocardiogram (ECG), electromyogram (EMG) of a trapezius muscle of the plurality of patients, and the pain-affect dataset may include merged image data and data collected from electrodermal activity (EDA), electrocardiogram (ECG), electromyogram (EMG) of a trapezius muscle of the plurality of patients.

In an additional embodiment, the present invention provides one or more non-transitory computer-readable media having computer-executable instructions for performing a method of running a software program on a computing device, the computing device operating under an operating system, the method including issuing instructions from the software program for performing pain level recognition in a patient of interest, which includes establishing a pain-affect dataset by merging a pain dataset comprising data acquired from a plurality of patients in response to a stimulus for eliciting pain with an affect dataset comprising data acquired from the plurality of patients in response to a stimulus to elicit a non-pain affect state in the plurality of patients. The media further includes instructions for training a neural network model using the established pain-affect dataset, monitoring a patient of interest with an image capture device to capture image data of a face of the patient of interest, collecting one or more biopotential signals of the patient of interest and applying the trained neural network model to the captured image data of the patient of interest and to the one or more biopotential signals collected from the patient of interest to identify a pain level of the patient of interest.

In another embodiment, the present invention provides a system for identifying a pain level of a patient of interest. The system includes, a video image capture device to capture image data of a patient of interest, one or more sensors to capture biopotential data of the patient of interest and processing circuitry configured as a neural network implementing a neural network based model to receive and process the image data and the biopotential data from the patient of interest to determine a pain level of the patient of interest, wherein the neural network is trained using a pain-affect database and wherein the pain-affect data is established by merging a pain dataset comprising data acquired from a plurality of patients in response to a stimulus for eliciting pain with an affect dataset comprising data acquired from the plurality of patients in response to a stimulus to elicit a non-pain affect state in the plurality of patients.

As such, in various embodiments, the present invention provides an improved system and method for the assessment of pain experienced by a patient in a real-world setting which takes into account a non-pain related affect state of the patient during the pain assessment.

### BRIEF DESCRIPTION OF THE DRAWINGS

The accompanying drawings, which are incorporated in and form a part of this specification, illustrate various embodiments and, together with the Description of Embodiments, serve to explain principles discussed below. The drawings referred to in this brief description should not be understood as being drawn to scale unless specifically noted.

FIG. 1. is an image illustrating an exemplary setup for image and biopotential data collection of a patient of interest.

FIG. 2A is a graphical illustration of a distribution of electrodermal activity (EDA) depicting affective states, wherein standardized (zero-mean and unit variance) data were sampled from one participant and illustrating that a subject could be in baseline (BL), low level pain (LLP), high level pain (HLP) and affect (A) states in different moments of time in the natural world.

FIG. 2B is a graphical illustration of a distribution of electrocardiogram (ECG) activity depicting affective states, wherein standardized (zero-mean and unit variance) data were sampled from one participant and illustrating that a subject could be in baseline (BL), low level pain (LLP), high level pain (HLP) and affect (A) states in different moments of time in the natural world.

FIG. 2C is a graphical illustration of a distribution of electromyogram (EMG) of a muscle of a patient of interest depicting affective states, wherein standardized (zero-mean and unit variance) data were sampled from one patient and illustrating that a patient could be in baseline (BL), low level pain (LLP), high level pain (HLP) and affect (A) states in different moments of time in the natural world.

FIG. 3A is a graphical illustration of a latent space representation of physiological data depicting affective states of one participant, using UMAP (uniform manifold approximation and projection) algorithm, wherein a grid of values was used to tune the major parameters (nearest neighbors=[2; 5; 10; 20; 50; 100; 200], and minimum distance=[0:0; 0:1; 0:25; 0:5; 0:8; 0:99]) of UMAP.

FIG. 3B is a graphical illustration of a latent space representation of physiological data depicting affective states of one specific demographic group, using UMAP (uniform manifold approximation and projection) algorithm, wherein a grid of values was used to tune the major parameters (nearest neighbors=[2; 5; 10; 20; 50; 100; 200], and minimum distance=[0:0; 0:1; 0:25; 0:5; 0:8; 0:99]) of UMAP.

FIG. 3C is a graphical illustration of a latent space representation of physiological data depicting affective states of all 62 participants, using UMAP (uniform manifold approximation and projection) algorithm, wherein a grid of values was used to tune the major parameters (nearest neighbors=[2; 5; 10; 20; 50; 100; 200], and minimum distance=[0:0; 0:1; 0:25; 0:5; 0:8; 0:99]) of UMAP.

FIG. 4A is a graphical illustration of the pain level (PL) recognition model performance on unknown participants in an investigated case including electrodermal activity (EDA) depicting affective states.

FIG. 4B is a graphical illustration of the pain level (PL) recognition model performance on unknown participants in an investigated case including electrocardiogram (ECG) activity depicting affective states.

FIG. 4C is a graphical illustration of the pain level (PL) recognition model performance on unknown participants in an investigated case including electromyogram (EMG) of a muscle of a patient of interest depicting affective states.

FIG. 4D is a graphical illustration of the pain level (PL) recognition model performance on unknown participants in an investigated case including electrodermal activity (EDA) depicting affective states, electrocardiogram (ECG) activity depicting affective states and electromyogram (EMG) of a muscle of a patient of interest depicting affective states.

FIG. 5A is a graphical illustration of the PL recognition model performance on unknown participants in an investigated case when demographic information was incorporated in feature vector as context and including electrodermal activity (EDA) depicting affective states.

FIG. 5B is a graphical illustration of the PL recognition model performance on unknown participants in an investigated case when demographic information was incorporated in feature vector as context and including electrocardiogram (ECG) activity depicting affective states.

FIG. 5C is a graphical illustration of the PL recognition model performance on unknown participants in an investigated case when demographic information was incorporated in feature vector as context and including electromyogram (EMG) of a muscle of a patient of interest depicting affective states.

FIG. 5D is a graphical illustration of the PL recognition model performance on unknown participants in an investigated case when demographic information was incorporated in feature vector as context and including electrodermal activity (EDA) depicting affective states, electrocardiogram (ECG) activity depicting affective states and electromyogram (EMG) of a muscle of a patient of interest depicting affective states.

FIG. 6A is a graphical illustration of the pain level (PL) recognition model performance on known participants in an investigated case including electrodermal activity (EDA) depicting affective states.

FIG. 6B is a graphical illustration of the pain level (PL) recognition model performance on known participants in an investigated case including electrocardiogram (ECG) activity depicting affective states.

FIG. 6C is a graphical illustration of the pain level (PL) recognition model performance on known participants in an investigated case including electromyogram (EMG) of a muscle of a patient of interest depicting affective states.

FIG. 6D is a graphical illustration of the pain level (PL) recognition model performance on known participants in an investigated case including electrodermal activity (EDA) depicting affective states, electrocardiogram (ECG) activity depicting affective states and electromyogram (EMG) of a muscle of a patient of interest depicting affective states.

FIG. 7A is a graphical illustration of the PL recognition model performance on known participants in an investigated case when demographic information was incorporated in feature vector as context and including electrodermal activity (EDA) depicting affective states.

FIG. 7B is a graphical illustration of the PL recognition model performance on known participants in an investigated case when demographic information was incorporated in feature vector as context and including electrocardiogram (ECG) activity depicting affective states.

FIG. 7C is a graphical illustration of the PL recognition model performance on known participants in an investigated case when demographic information was incorporated in feature vector as context and including electromyogram (EMG) of a muscle of a patient of interest depicting affective states.

FIG. 7D is a graphical illustration of the PL recognition model performance on known participants in an investigated case when demographic information was incorporated in feature vector as context and including electrodermal activity (EDA) depicting affective states, electrocardiogram (ECG) activity depicting affective states and electromyogram (EMG) of a muscle of a patient of interest depicting affective states.

FIG. 8A is a graphical illustration of the personalized PL recognition model performance in investigated cases, wherein each point in each box-whisker plot indicates F1-macro computed on each participant and including electrodermal activity (EDA) depicting affective states.

FIG. 8B is a graphical illustration of the personalized PL recognition model performance in investigated cases, wherein each point in each box-whisker plot indicates F1-macro computed on each participant and including electrocardiogram (ECG) activity depicting affective states.

FIG. 8C is a graphical illustration of the personalized PL recognition model performance in investigated cases, wherein each point in each box-whisker plot indicates F1-macro computed on each participant and including electromyogram (EMG) of a muscle of a patient of interest depicting affective states.

FIG. 8D is a graphical illustration of the personalized PL recognition model performance in investigated cases, wherein each point in each box-whisker plot indicates F1-macro computed on each participant and electrodermal activity (EDA) depicting affective states, electrocardiogram (ECG) activity depicting affective states and electromyogram (EMG) of a muscle of a patient of interest depicting affective states.

### DETAILED DESCRIPTION OF THE INVENTION

Reference will now be made in detail to various embodiments, examples of which are illustrated in the accompanying drawings. While various embodiments are discussed herein, it will be understood that they are not intended to be limiting. On the contrary, the presented embodiments are intended to cover alternatives, modifications, and equivalents, which may be included within the spirit and scope of the various embodiments as defined by the appended claims. Furthermore, in this Detailed Description of the Invention, numerous specific details are set forth in order to provide a thorough understanding. However, embodiments may be practiced without one or more of these specific details. In other instances, well known methods, procedures, components, and circuits have not been described in detail as not to unnecessarily obscure aspects of the described embodiments.

It will be understood that, although the terms first, second, third, etc., may be used herein to describe various elements, components, regions, layers, and/or sections, these elements, components, regions, layers, and/or sections should not be limited by these terms. These terms are only used to distinguish one element, component, region, layer, or section from another region, layer, or section. Thus, a first element, component, region, layer, or section discussed below could be termed a second element, component, region, layer, or section without departing from the teachings of the present invention.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In various embodiments, the present invention proposes that affect be accounted for when assessing pain to provide a robust assessment in real-world settings, given that the model of the invention is aware of other affective states. A new pain-affect dataset is provided and referred to as to as bioVid pain-affect, which allows for the development of a reliable and robust pain assessment model that considers multiple affect states commonly experienced by patients (e.g., sadness and fear) in clinical settings. The experimental results on the pain-affect dataset indicate that pain assessment models that do not take affect into consideration are likely to drift and achieve poor inference when applied to real-world settings.

The embodiments of the present invention enhance current assessment practices by accounting for non-pain affect when recognizing pain. It has been shown that patients in clinical settings undergo a wide range of affect states including anxiety, comfort, anger, and sadness. This could lead to current models failing when deployed in clinical settings. Additionally, the present invention also provides adjustments to pain score/intensity based on the underlying affect of the patient. It has been shown that there is altered pain sensitivity in patients who experience pre-existing anxiety about pain. It has also been shown that depressed patients tend to report higher pain score as they have higher pain sensitivity as compared to non-depressed patients. Similarly, it has been shown that inducing “joy” can result in a significantly lower pain scores reported by patients.

The proposed inventive concepts can provide a better understating of patients' pain experience, which is an important step in improving their clinical outcomes and personalized pain assessment, accounting for underlying affect states of each patient, can lead to the development of personalized treatment plans for each patient.

Previous pain assessment models might not be clinically useful as they are trained to generally distinguish pain from neutral states without considering individual characteristics and underlying affect. Further, neglecting psychological states (e.g., distress) while assessing pain and developing treatment plans can lead to serious outcomes.

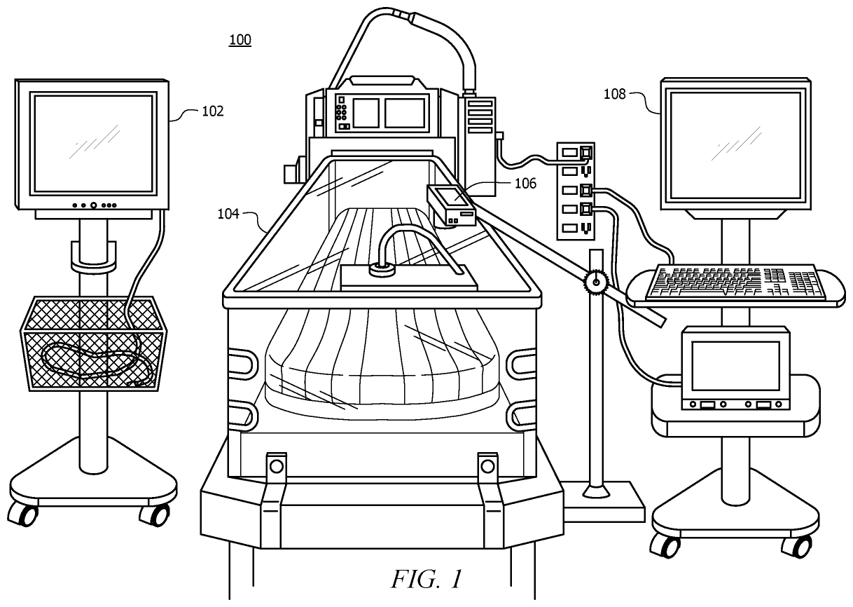

As shown in FIG. 1, a system 100 for identifying a pain level of a patient of interest may include a video image capture device 105 for capturing real-time video data of the patient of interest. In a particular, embodiment, the patient of interest may be a neonate positioned in an incubator 115, or alternatively a crib or bed. The system 100 may further include a monitoring device 110 for displaying the video captured by the video image capture device 105. The video image capture device 105 would be placed above the patient of interest in a manner to allow for the capture of facial expressions from the neonate patient

In some embodiments the video image capture device 105 may be a GoPro Hero4+video camera which can be used to record video and audio signals. The camera may be triggered remotely using a GoPro application installed on a smartphone or alternatively on the monitoring device 110. The capture image data includes the patient's face, head, and body as well as the sounds from the patient and background noise (e.g., sounds of equipment and nurses).

The system 100 may additionally include a data monitoring device 102 for collecting biopotential data from the patient of interest via a plurality of biosensors (not depicted). The biosensors, in combination with, the data monitoring device 102 are configured to collect and store one or more biopotential signals, which may include electrodermal activity (EDA), electrocardiogram (ECG) activity, electromyogram (EMG) of a trapezius muscle, EMG of a corrugator muscle and EMG of a zygomaticus muscle of a patient of interest.

The system further includes processing circuitry 108 configured as a neural network implementing a neural network based model to receive and process the image data and the biopotential data and to determine a pain level of the patient of interest, wherein the neural network is trained using a pain-affect database, as will be described in more detail below.

In accordance with the present invention, the video images and biopotential signals obtained by the system illustrated in FIG. 1 are provided an input to a neural network model for automatically identifying a pail level of the patient of interest, as described in more detail below.

In various embodiments, a new dataset (bioVid pain-affect) has been curated by merging publicly available bioVid pain and bioVid emotion datasets. In one embodiment, the bioVid pain dataset contains two sessions of recording participants pain and baseline states and the bioVid emotion dataset contains one session of recording participants discrete affect states including amusement, anger, disgust, fear, sadness. The data was collected in the same lab settings using similar equipment. Participants were within the age range of 19 to 64 years and in total there were 91 subjects, including 45 females and 46 males, across both datasets.

To elicit spontaneous pain, in the bioVid pain dataset, self-calibrated heat was applied as the stimulus. There are five pain levels in the bioVid pain dataset including, baseline (BL), pain level (PL)=0, pain threshold−PL=1 and pain tolerance level−PL=4. After collecting data from the participants, two intermediate pain levels were selected: PL=2 and PL=3. In the bioVid emotion dataset, clips from videos were used to elicit spontaneous discrete emotions including amusement (Am), anger (An), disgust (Di), fear (F) and sadness (S) in the patients. To merge the 2 datasets, the common subjects were selected that appeared in both the bioVid pain and the bioVid emotion datasets, resulting in 82 subjects in total. 20 participants were then removed from the merged dataset because they did not show pain responses during the pain stimulus, resulting in 62 participants, 33 females and 29 males. Merging of the datasets resulted in a modified age range of 20 to 65 with a median age of 36.

The bioVid emotion dataset contains three biopotential signals: (1) skin conductance level or electrodermal activity (EDA), (2) electrocardiogram (ECG) and (3) electromyogram (EMG) of trapezius muscle, and videos of participants' frontal face. In addition to the above-mentioned modalities, bioVid pain dataset contains EMG signal collected from corrugator and zygomaticus muscles. In the merged bioVid pain-affect dataset, only the common biopotential signals (e.g., EDA, ECG, EMG from trapezius muscle) and videos were selected. This work focused only on the biopotential signals, as studies in healthcare indicated that biopotential signals are major objective indicators of pain and other affect states.

The bioVid pain dataset contains 5.5 seconds long subsamples (from the raw data) along with the raw data, while the bioVid emotion dataset only contains raw data having a range between 32 and 245 seconds long. In this work, we extracted 5.5 seconds long subsamples from the bioVid raw emotion data following the pain dataset to have the same length samples for pain, baseline and affect in our merged dataset.

The visual responses of PL 1 and PL 2 in bioVid pain dataset were difficult to distinguish due to the similarity in terms of responses and some subjects not showing any affective responses. Further, PL 3 and PL 4 had similar responses to a certain extent. Based on those findings, in this new merged dataset, PL 1 and PL 2 were merged to one level, named low level pain (LLP), and PL 3 and PL 4 were merged to another pain level, named high level pain (HLP). Since the primary goal is to investigate the influence of affect in PL recognition approaches, all discrete affects (e.g., Amusement (Am), Sadness (Sa), Anger (An), Disgust (Di) and Fear (Fe)) were merged to one specific affect class named Affect (A). Hence, in the new bioVid pain-affect dataset, there are four discrete classes: Baseline (BL), Low Level Pain (LLP), High Level Pain (HLP), and Affect (A).

The focus of the present invention is to recognize pain level while taking into account affect states that exist in real-world settings such as anger and anxiety. Recall that one considers discrete affect states (e.g., amusement, sadness, fear), and combines these states to one affect state named A. It is hypothesized that the consideration of different affect states while assessing pain is necessary to obtain accurate, transparent, and ethical PL recognition models that are expected to deploy in sensitive domains such as healthcare. To do so, it is proposed to incorporate affect in the pain level (PL) recognition model as a category (e.g., A in the studied dataset) along multiple pain levels (e.g., LLP, HLP in the studied dataset). Depending on the context, BL category could be incorporated in affect category, as BL is likely to be a neutral/relaxed affect state, and for some subjects relaxed states could be other positive or negative affect states (e.g., amusement, fear) depending on the situation, environment or time.

To create a feature vector for a given sample, the signals (e.g., EDA) were down-sampled by computing the moving average using a sliding window with 80% overlap. Before down-sampling the signals, a filter was used to remove noise from the raw signals. Each signal in the range [0, 1] was then normalized using a min-max normalization method. A normalized feature vector was then created as fₘ=[c₁, c₂, . . . cₙ], where c and n are the down-sampled signal frames/components and length of down-sampled signal, respectively.

Since context is an essential component for modeling of affective computing task, a feature vector was also created incorporating gender, age, and demographic group of participants. Demographic group is constructed via grouping participants based on their age, and gender. Females aged in between (20, 30), (30, 50), and (50, 65) are grouped and labeled as F1; F2; F3, respectively. Males in similar age categories are grouped and labeled as M4; M5; M6.

In case −1, naive PL recognition approaches were explored, in which only baseline and pain data were used during training, validation and testing data. This case represents the naive approach used currently in PL recognition studies.

In case 0, real-world settings were simulated on the naive PL recognition case −1 approach. As previously discussed, a person is likely to experience pain, baseline, and other affects in real-world settings. Hence, the PL recognition model developed in case −1 (pain and baseline only) is reproduced, and the model is tested assuming the affect exists in the testing dataset.

Case 5 is based on the proposal of the present invention. In this case, it is acknowledged during the development of PL recognition model and during testing in real-world settings, that a person would experience affect (e.g., sadness, fear), baseline, and multiple levels of pain. In the training, validation, and testing dataset, both affect, and pain data are included. In the affect data, the baseline state is incorporated assuming it is part of affect state.

Case 6 is a variation of case 5 in which the baseline data is removed from other affect data. This case allows one to investigate the performance of PL recognition model in absence of baseline, and in presence of other affect states. Also, in this case, one can also investigate the impact of a balanced class distribution among classes (e.g., LLP, HLP, and A) in the training dataset. Contrary to cases −1, 0 and 5, here the training dataset uniformity is preserved in terms of number of examples and class proportion to ensure fair evaluation.

Since the major goal of this study is to investigate the limitation of previous PL recognition approach and to evaluate the effectiveness of proposed approach, three types of model validations were performed: i) Evaluation on Unknown/New Participants: used leave-n-participant-out validation, where n=15 in the experiments to evaluate all studied cases on participants who are unknown during training; ii) Evaluation on Known/Partially Known Participants: randomly split the whole dataset into 70% training and 30% test data, and the experiments were run 5 times with different seeds; iii) Person-Specific Evaluation: this evaluation, trained and tested on each participant under the assumption that participants are different in terms of eliciting affective behavior, hence, for a given participant's data, the data was split into 70% training and 30% test data, and the experiments were run 5 times with different seeds. F1 macro (macro-average F1 measure) was used to report the model performance given its robustness towards balanced and unbalanced dataset.

To investigate the performance of PL recognition models in the above-mentioned cases, PL recognition models were built in unimodel and multimodel settings, i.e., trained and tested PL recognition model on EDA, ECG, EMG separately, and their combination (EDA+ECG+EMG). Experiments were also performed by combining each of the modalities with demographic information and combining the demographic information with the fused modalities (EDA+ECG+EMG). For PL recognition algorithm, experiments were done with three well-known and well-understood classification algorithms such as K Nearest Neighbors (KNN), Random Forest (RF), and Extreme Gradient Boosting (XGB). The classifiers were tuned empirically. In the case of KNN, the number of neighbor parameter was set to 5. In the case of RF and XGB, the number of the tree estimator parameter was set to 750, given that more estimators are likely to produce better results. Finally, in XGB, the learning rate was set to 0.1.

To the best of the inventor's knowledge, previous studies have failed to account for affect states in pain intensity/level recognition. Therefore, there are no state-of-the-art works to compare the present invention against. Considering this, this work can serve as a baseline in this regard.

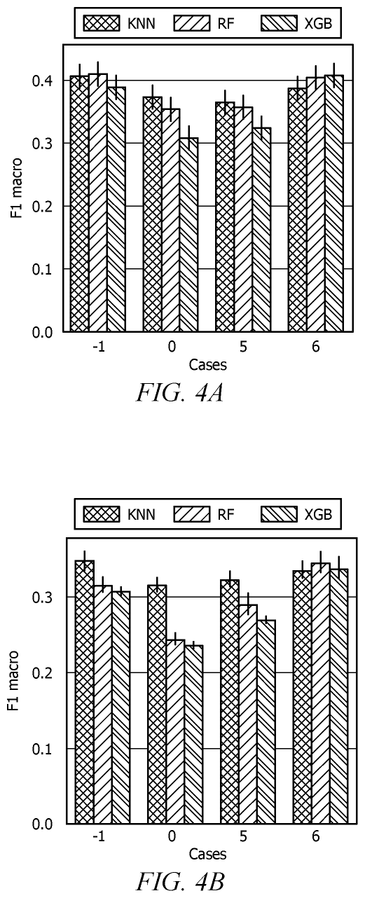

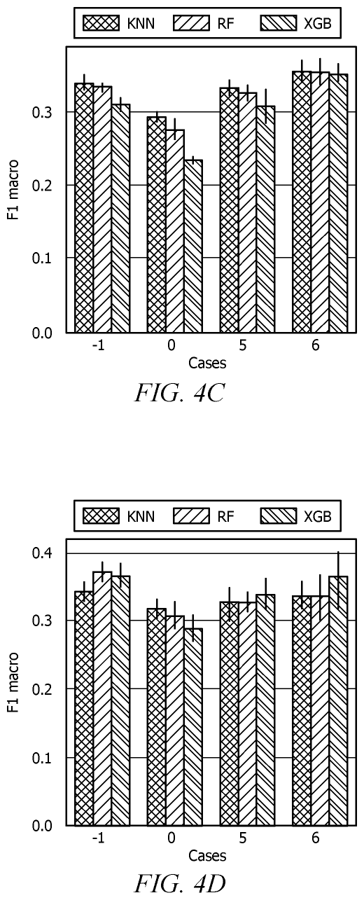

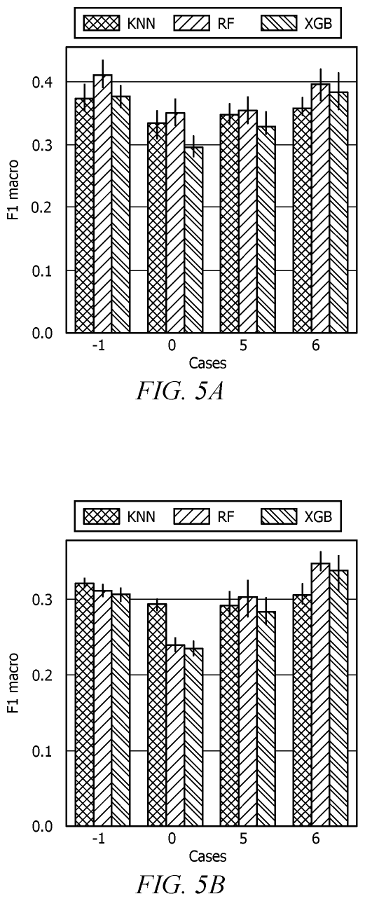

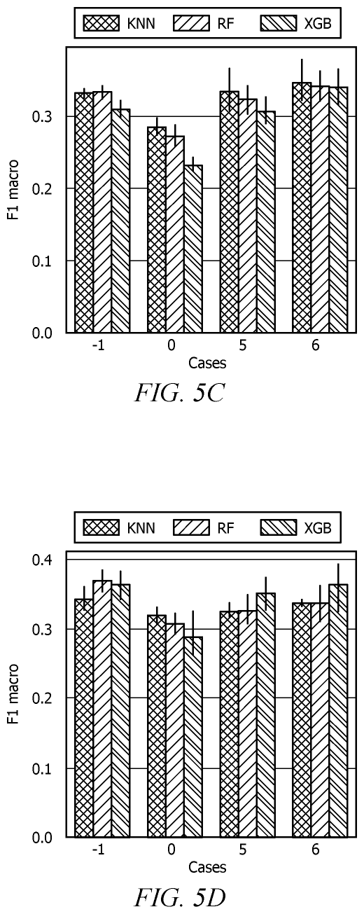

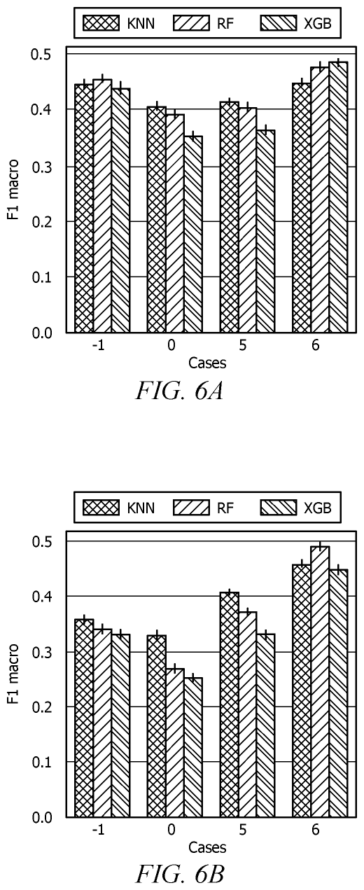

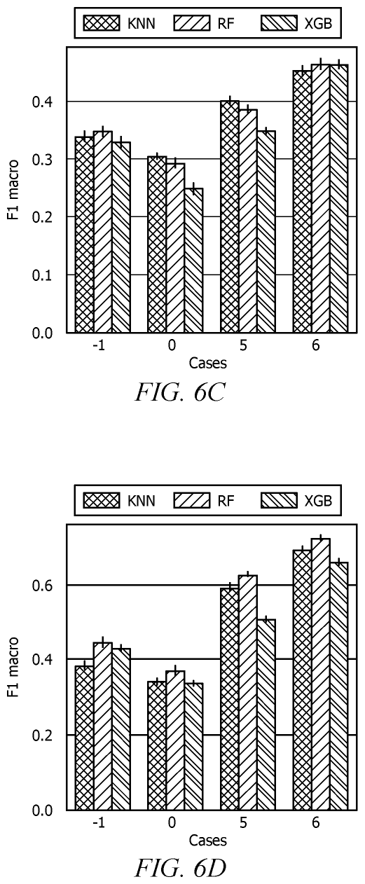

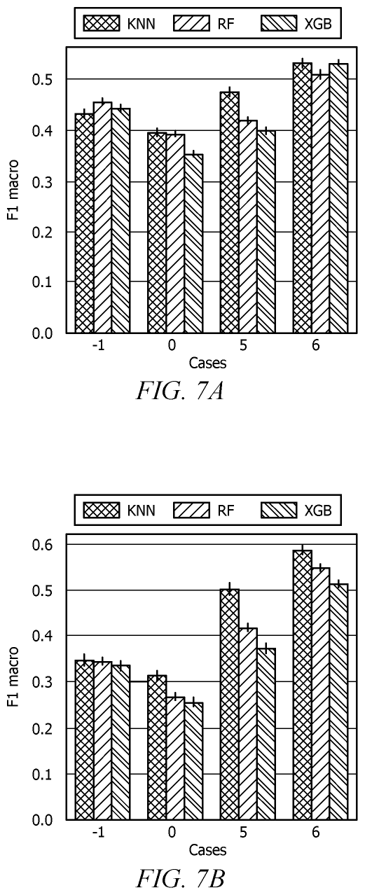

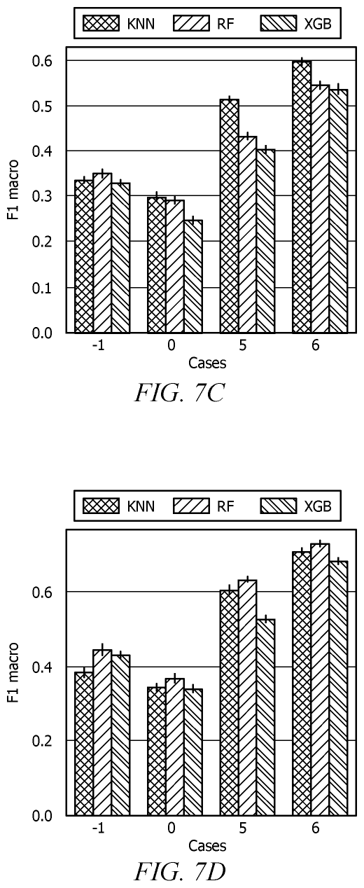

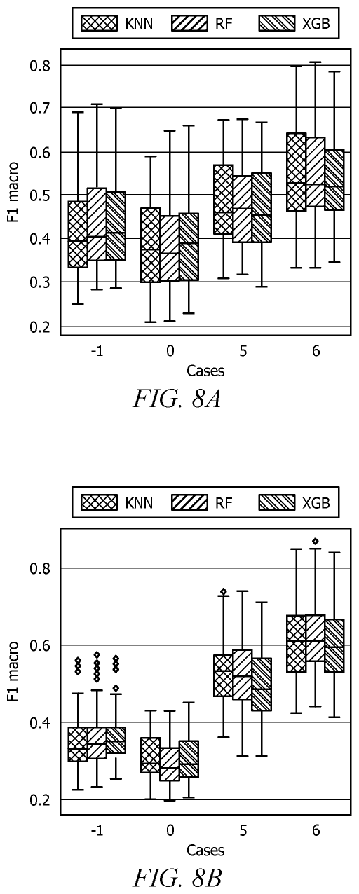

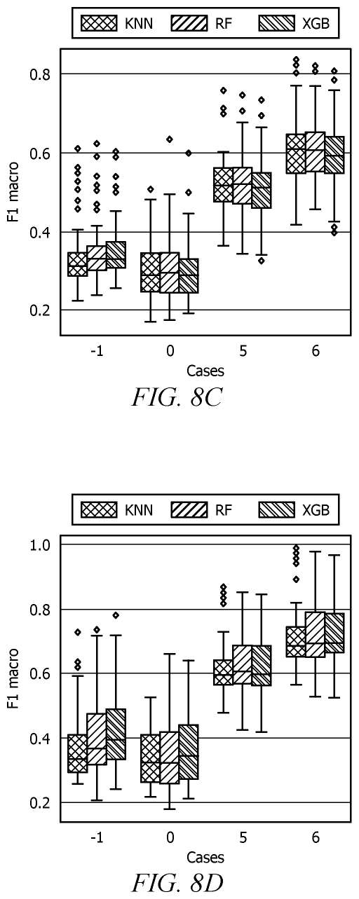

Recall that in case −1 and case 0, the naive PL recognition model was trained, i.e., model does not know about the affect states (e.g., anger, sadness). Also, training data size and class proportion were kept uniform in the −1, 0 and 5 cases, for fair evaluation. When the naive PL recognition model was tested in case −1, by simulating the existence of affects, the model showed drift in terms of recognition rate compared to the results obtained in case −1, as can be seen in FIG. 4A-FIG. 4C, FIG. 5A-FIG. 5D, FIG. 6A-FIG. 6D, FIG. 7A-FIG. 7D and FIG. 8A-FIG. 8D. This can be explained, in part, due to the PL recognition model not being aware of the affect data during training.

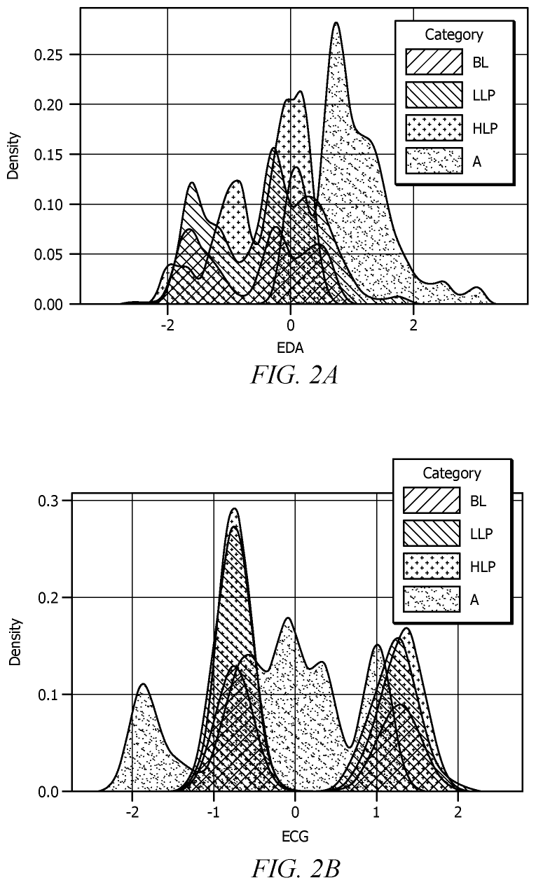

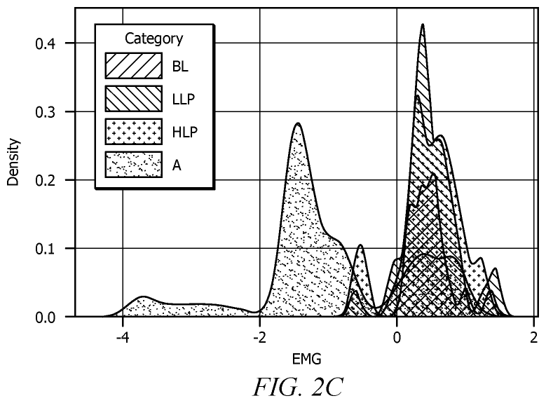

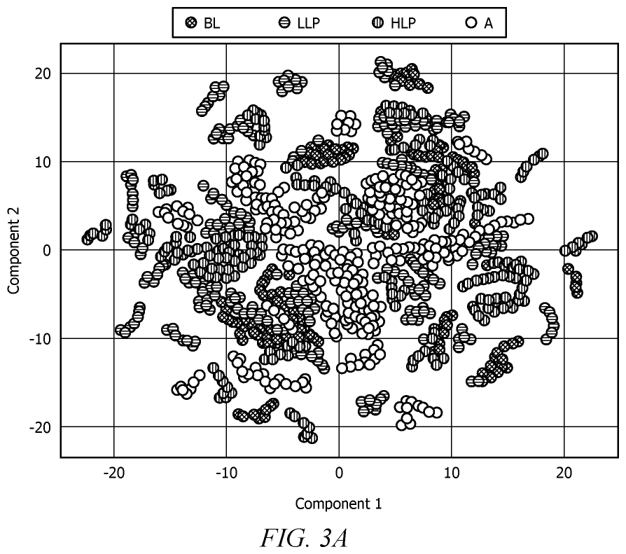

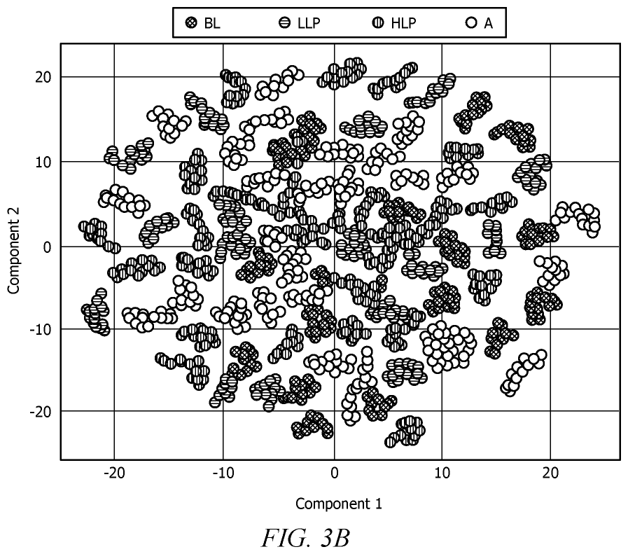

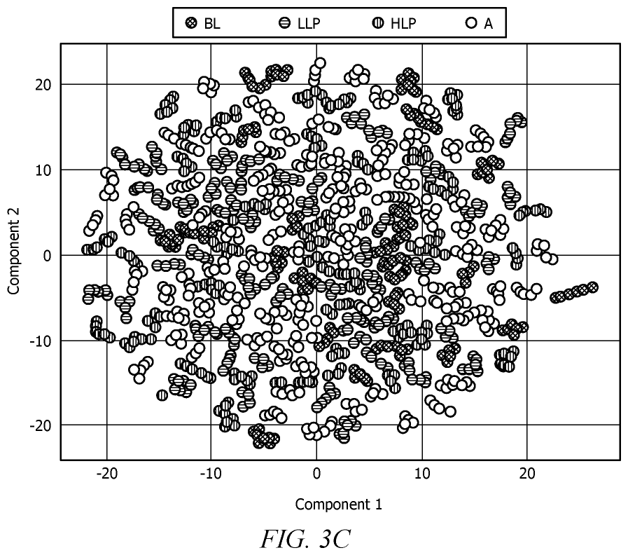

Some evidence behind this issue could be found from FIG. 2A-FIG. 2C and FIG. 3A-FIG. 3C, wherein FIG. 2A-FIG. 2C illustrates that a patient of interest could be in BL, LLP, HLP an A states in different moments of time in the natural world and FIG. 3A-FIG. 3C illustrates variations in latent space representation of physiological data depicting affective states depending upon whether the data is sample from one participant, a specific demographic group or all available participants. Since affect data is not considered in the naive modeling case, it is extremely hard for the naive PL recognition model to generalize or adapt to the affect data. More precisely, during inference in simulated natural world, the trained naive PL recognition model seemed to treat the affect data as unknown data.

In case 5, affect is taken into account in the PL recognition model of the present invention. As can be seen in FIG. 4A-FIG. 4C, FIG. 5A-FIG. 5D, FIG. 6A-FIG. 6D, FIG. 7A-FIG. 7D and FIG. 8A-FIG. 8D, the model of the present invention showed significant improvement in terms of recognition performance, when compared to case 0. Recall that the test dataset is the same in both case 5 and case 0. This can be explained, in part, due to the affect distribution being known to the proposed PL recognition model.

Recall that case 6 was designed to investigate the influence of balanced training dataset in terms of class proportion. Also, to evaluate the proposed model in the absence of baseline (BL) data in both the training and test dataset. In this case, an improvement in recognition performance is seen, which can be explained, in part, by the even distribution of LLP, HLP and A categories in the training dataset.

The following experiments were performed to evaluate whether one could build a generalizable PL recognition model that can generalize to everyone, as people are likely to be different in terms of eliciting affects. Comparing FIG. 6A-FIG. 6D, which considers the data to be from known participants to FIG. 4A-FIG. 4D, which considers the data to be from unknown participants, and FIG. 7A-FIG. 7D, which considers the data to be from known participants, to FIG. 5A-FIG. 5D, which considers the data to be from unknown participants, it can be seen that when it is assumed participants are known, improvement in performance is seen, this could be due to the subjective variability. In FIG. 3A-FIG. 3D, it can be seen that it is easier to distinguish between BL, LLP, HLP, and A in individual cases (FIG. 3A), however, sampled from multiple people (FIG. 3C), it is harder to distinguish. An interesting pattern can be observed is that as the number of subjects in the samples is increased, it gets more and more difficult to distinguish between affective states, which in turn is likely to make generalization to unknown participants harder. Demographic information as context to evaluate the influence of gender, age and demographic group was also analyzed and the results suggest that demographic information showed some improvement in PL recognition performance in known participants setting, as presented in FIG. 5A-FIG. 5D and FIG. 7A-FIG. 7D.

FIG. 8A-FIG. 8D shows that the PL recognition model gave encouraging results for some participants, however, for others even though the models were personalized by training and testing on each participant, it is challenging to distinguish between multiple affect states. There could be multiple explanations for this. First, it is possible those participants did not feel affect or pain during experiments. Secondly, they may have been going through some other affective state that was not captured in the data. Lastly, the physiological data was not different across the affective states for those participants (i.e., pain and affect looked similar). Considering this, it is necessary to further look into the data to make any concluding remarks. Finally, in all evaluations (unimodal and multimodal features with/without incorporation of context), improvement was observed when affect was accounted for in PL recognition approaches compared to cases when affect states were not accounted for.

In this work, the necessity of including non-pain affect in pain level recognition was investigated, which is a current limitation. Using just a baseline (in most cases which is normally neutral affect state) during PL recognition modeling (case −1 in the case studies) produced reasonable results in a controlled environment. However, as evidenced from the experiments, these naive models may fail in natural settings as people experience a diverse set of affect states, apart from neutral and pain. Based on these experiments, the various examples of the present invention solve the problem by incorporating affect into pain level recognition approaches.

As can be seen in pain level recognition when accounting for affect is a challenging task due to the similarity of data patterns of pain and non-pain affect, as well as the variability of data based on people differences. The proposed formulation of the present invention still performed comparatively better due to its ability to adapt to the affect dataset. Hence, even though incorporation of affect could make the modeling challenging, it would make the modeling sound, reliable, and realistic. The main limitation of this study is that it is limited to physiological signals only. In future work, behavioral data can be explored, such as face images and facial action units, along with the combination of physiological and behavioral data.

The present invention may be embodied on various computing platforms that perform actions responsive to software-based instructions and most particularly on touchscreen portable devices. The following provides an antecedent basis for the information technology that may be utilized to enable the invention.

The computer readable medium described in the claims below may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any non-transitory, tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. However, as indicated above, due to circuit statutory subject matter restrictions, claims to this invention as a software product are those embodied in a non-transitory software medium such as a computer hard drive, flash-RAM, optical disk or the like.

Program code embodied on a computer readable medium may be transmitted using any appropriate medium, including but not limited to wireless, wire-line, optical fiber cable, radio frequency, etc., or any suitable combination of the foregoing. Computer program code for carrying out operations for aspects of the present invention may be written in any combination of one or more programming languages, including an object oriented programming language such as Java, C #, C++, Visual Basic or the like and conventional procedural programming languages, such as the “C” programming language or similar programming languages.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

It should be noted that when referenced, an “end-user” is an operator of the software as opposed to a developer or author who modifies the underlying source code of the software. For security purposes, authentication means identifying the particular user while authorization defines what procedures and functions that user is permitted to execute.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween.

What is claimed is:

1. A computer-implemented method for identifying a pain level of a patient of interest, the method comprising:
establishing a pain-affect dataset by merging a pain dataset comprising data acquired from a plurality of patients in response to a stimulus for eliciting pain with an affect dataset comprising data acquired from the plurality of patients in response to a stimulus to elicit a non-pain affect state in the plurality of patients;
training a neural network model using the established pain-affect dataset;
monitoring a patient of interest with an image capture device to capture image data of a face of the patient of interest;
collecting one or more biopotential signals of the patient of interest; and
applying the trained neural network model to the captured image data of the patient of interest and to the one or more biopotential signals collected from the patient of interest to identify a pain level of the patient of interest.

2. The method of claim 1, wherein the non-pain affect state is selected from amusement, anger, disgust, fear and sadness.

3. The method of claim 1, wherein the pain dataset further comprises data acquired from the plurality of patients in response to no stimulus for eliciting pain to establish a baseline for the pain dataset.

4. The method of claim 1, wherein the pain dataset is a bioVID pain dataset.

5. The method of claim 4, wherein the bioVid pain dataset comprises face image data and data collected from one or more biopotential signals of the plurality of patients.

6. The method of claim 5, wherein the one or more biopotential signals are selected from electrodermal activity (EDA), electrocardiogram (ECG), electromyogram (EMG) of a trapezius muscle, EMG of a corrugator muscle and EMG of a zygomaticus muscle of the plurality of patients.

7. The method of claim 1, wherein the non-pain affect dataset is a bioVid emotion dataset.

8. The method of claim 7, wherein the bioVid emotion dataset comprising face image data and data collected from one or more biopotential signals of the plurality of patients.

9. The method of claim 8, wherein the biopotential signals are selected from electrodermal activity (EDA), electrocardiogram (ECG), electromyogram (EMG) of a trapezius muscle of the plurality of patients.

10. The method of claim 1, wherein the pain dataset is a bioVid pain dataset comprising image data and data collected from electrodermal activity (EDA), electrocardiogram (ECG), electromyogram (EMG) of a trapezius muscle, EMG of a corrugator muscle and EMG of a zygomaticus muscle of the plurality of patients, the non-pain affect dataset is a bioVid affect dataset comprising image data and data collected from electrodermal activity (EDA), electrocardiogram (ECG), electromyogram (EMG) of a trapezius muscle of the plurality of patients, and wherein the pain-affect dataset comprises merged image data and data collected from electrodermal activity (EDA), electrocardiogram (ECG), electromyogram (EMG) of a trapezius muscle of the plurality of patients.

11. The method of claim 1, wherein the one or more biopotential signals collected from the patient of interest are selected from electrodermal activity (EDA), electrocardiogram (ECG) and electromyogram (EMG) of a trapezius muscle of the patient of interest.

12. One or more non-transitory computer-readable media having computer-executable instructions for performing a method of running a software program on a computing device, the computing device operating under an operating system, the method including issuing instructions from the software program comprising:
establishing a pain-affect dataset by merging a pain dataset comprising data acquired from a plurality of patients in response to a stimulus for eliciting pain with an affect dataset comprising data acquired from the plurality of patients in response to a stimulus to elicit a non-pain affect state in the plurality of patients;
training a neural network model using the established pain-affect dataset;
monitoring a patient of interest with an image capture device to capture image data of a face of the patient of interest;
collecting one or more biopotential signals of the patient of interest; and
applying the trained neural network model to the captured image data of the patient of interest and to the one or more biopotential signals collected from the patient of interest to identify a pain level of the patient of interest.

13. The media of claim 12, wherein the non-pain affect state is selected from amusement, anger, disgust, fear and sadness.

14. The media of claim 12, wherein the pain dataset is a bioVID pain dataset.

15. The media of claim 14, wherein the non-pain affect dataset is a bioVid emotion dataset.

16. A system for identifying a pain level of a patient of interest, the system comprising:
a video image capture device to capture image data of a patient of interest;
one or more sensors to capture biopotential data of the patient of interest; and
processing circuitry configured as a neural network implementing a neural network based model to receive and process the image data and the biopotential data from the patient of interest to determine a pain level of the patient of interest, wherein the neural network is trained using a pain-affect database and wherein the pain-affect data is established by merging a pain dataset comprising data acquired from a plurality of patients in response to a stimulus for eliciting pain with an affect dataset comprising data acquired from the plurality of patients in response to a stimulus to elicit a non-pain affect state in the plurality of patients.

17. The system of claim 14, wherein the non-pain affect state is selected from amusement, anger, disgust, fear and sadness.

18. The system of claim 14, wherein the pain dataset is a bioVID pain dataset.

19. The system of claim 14, wherein the non-pain affect dataset is a bioVid emotion dataset.

20. The system of claim 14, wherein the pain dataset is a bioVid pain dataset comprising image data and data collected from electrodermal activity (EDA), electrocardiogram (ECG), electromyogram (EMG) of a trapezius muscle, EMG of a corrugator muscle and EMG of a zygomaticus muscle of the plurality of patients, the non-pain affect dataset is a bioVid affect dataset comprising image data and data collected from electrodermal activity (EDA), electrocardiogram (ECG), electromyogram (EMG) of a trapezius muscle of the plurality of patients, and wherein the pain-affect dataset comprises merged image data and data collected from electrodermal activity (EDA), electrocardiogram (ECG), electromyogram (EMG) of a trapezius muscle of the plurality of patients.
